(19) United States
(12) Patent Application Publication
(10) Pub. No.: US 2026/0347836 A1
(43) Pub. Date: Nov. 5, 2026

(54) DIRECTED ACETYLATION OF MRNA THROUGH ENGINEERED SNORNA ADAPTERS

(71) Applicant: The United States of America, as represented by the Secretary, Dept. of Health and Human Services, Bethesda, MD (US)

(72) Inventors: Shalini Oberdoerffer, Bethesda, MD (US); Sarah Schiffers, Bethesda, MD (US)

(21) Appl. No.: 19/116,059

(22) PCT Filed: Nov. 6, 2023
(86) PCT No.: PCT/US2023/078830

§ 371 Date: Mar. 27, 2025

Related U.S. Application Data
(60) Provisional application No. 63/423,714, filed on Nov. 8, 2022.

(51) Int. Cl. C12N 15/11; A61K 31/7105

(52) U.S. Cl. CPC C12N 15/111; A61K 31/7105; C12N 2310/11; C12N 2310/3519

(57) ABSTRACT

The disclosure provides engineered nucleotide sequences, vectors, and pharmaceutical compositions that can be used in methods for treating a disease or disorder with an associated haploinsufficiency and/or cancer.

### CROSS-REFERENCE TO RELATED APPLICATIONS

This application claims the benefit of U.S. Provisional Application No. 63/423,714, filed Nov. 8, 2022, which is incorporated by reference herein in its entirety.

### GOVERNMENT RIGHTS

This invention was made with Government support. The National Cancer Institute and the National Institutes of Health funded the subject matter of this disclosure (ZIA BC 011239). The Federal Government has certain rights to this invention.

### REFERENCE TO AN ELECTRONIC SEQUENCE LISTING

The instant application contains an electronic Sequence Listing that has been submitted electronically and is hereby incorporated by reference in its entirety. The sequence listing was created on Nov. 6, 2023, is named “22-1953-WO_Sequence-Listing.xml” and is 9,934 bytes in size.

### BACKGROUND OF THE INVENTION

### Field of the Disclosure

This disclosure provides compositions and methods related to directed acetylation by engineered snoRNA molecules.

### Description of Related Art

Small nucleolar RNAs (snoRNAs) are non-coding RNAs that primarily accumulate in the nucleoli and typically comprise about 60 to 300 nucleotides. Based on the characteristic nucleotide motifs and association with canonical partner proteins, snoRNAs are classified into either C/D- (SNORD) or H/ACA-box (SNORA) subfamilies. Eukaryotic box C/D RNAs are typically 70 to 120 nucleotides in length and contain two conserved motif elements: box C (RUGAUGA; wherein R is a purine) and box D (CUGA) located at the 5′- and 3′-termini of the RNA molecule, respectively (see FIG. 1). Human N-acetyltransferase 10 (NAT10) acetylates RNA through association with snoRNA guides. NAT10 activity can be manipulated to develop novel therapeutics for specific defects in mRNA translation. Traditional gene therapy approaches rely on eliminating deleterious transcripts. However, strategies that involve genome manipulation carry inherent risks and ablation does not address diseases that result from inadequate mRNA translation. Thus, there is a need for treatment of any disease or disorder where moderate protein expression can lead to alleviation of disease phenotype (i.e., a disease or disorder with an associated haploinsufficiency). Provided herein are tools and methods that function in human cells for precise RNA epigenetic engineering. The compositions and methods provided herein can modulate gene regulation, target diseases, and serve as the basis of curative therapies for many previously untreatable diseases and/or disorders.

### SUMMARY OF THE INVENTION

snoRNA-guided acetylation of select mRNA. The inventors examined the feasibility of capitalizing on NAT10's property to direct acetylation, particularly the N4-acetylcytidine (ac4C) modification, to specific transcript substrates to regulate protein synthesis. As reported herein, engineered chimeric snoRNA guides can recruit NAT10 to a target transcript that causes directed acetylation of the target transcript, which regulates protein production. As a proof of principle, the inventors generated a chimeric snoRNA composed of the Box C/D snoRNA SNORD13 and antisense elements directed to, for example, GAPDH. The snoRNA was delivered into HeLa cells through incorporation into a minigene vector. Recovery of GAPDH transcripts and mass spectrometry confirmed successful targeted acetylation in human cells lines. As further described below, this disclosure provides directed acetylation through engineered snoRNA adapters and can be useful for the treatment of any disease or disorder where moderate protein expression can lead to alleviation of disease phenotype (i.e., a disease or disorder with an associated haploinsufficiency).

It is against the above background that the present invention provides certain advantages over the prior art.

In one aspect, this disclosure provides an engineered nucleotide sequence comprising, 5′ to 3′:
(a) a first targeting sequence;
(b) a C box motif containing sequence;
(c) a second targeting sequence; and
(d) a D box motif containing sequence.

In certain embodiments of the engineered nucleotide sequence disclosed herein, the first targeting sequence comprises about 15 to about 30 nucleotides.

In some embodiments of the engineered nucleotide sequence disclosed herein, the second targeting sequence comprises about 15 to about 30 nucleotides.

In some embodiments of the engineered nucleotide sequence disclosed herein, the first targeting sequence and the second targeting sequence are complementary to separate regions of a target sequence.

In some embodiments of the engineered nucleotide sequence disclosed herein, the target sequence comprises an RNA transcript.

In some embodiments of the engineered nucleotide sequence disclosed herein, the target sequence of the RNA transcript comprises a 5′ untranslated region (UTR) of the RNA transcript.

In some embodiments of the engineered nucleotide sequence disclosed herein, the target sequence of the RNA transcript comprises a coding sequence (CDS) of the RNA transcript.

In some embodiments of the engineered nucleotide sequence disclosed herein, the target sequence comprises a known acetylation site or a putative acetylation site.

In some embodiments of the engineered nucleotide sequence disclosed herein, the target sequence is within about 30-50 nucleotides of a known acetylation site or a putative acetylation site.

In some embodiments of the engineered nucleotide sequence disclosed herein, the C box motif containing sequence comprises a 5′ RUGAUGA-3′ sequence (wherein R is a purine).

In some embodiments of the engineered nucleotide sequence disclosed herein, the C box motif containing sequence comprises about 40 to about 200 nucleotides.

In some embodiments of the engineered nucleotide sequence disclosed herein, the D box motif containing sequence comprises a 5′ CUGA-3′ sequence.

In some embodiments of the engineered nucleotide sequence disclosed herein, the D box motif containing sequence comprises about 15 to about 50 nucleotides.

In some embodiments of the engineered nucleotide sequence disclosed herein, the engineered nucleotide sequence recruits NAT10 to the target sequence and causes acetylation (e.g., N4-acetylcytidine (ac4C) modification) of the target sequence.

In some embodiments of the engineered nucleotide sequence disclosed herein, the targeting sequences are complementary to a target sequence in a desired target gene, and the gene is involved or implicated in any disease or disorder where moderate protein expression can lead to alleviation of the disease or disorder phenotype.

In some embodiments of the engineered nucleotide sequence disclosed herein, the desired target gene encodes a ribosomal protein, a gene involved in RNA processing, a gene involved in gene transcription and/or translation, or a gene involved in proteostasis.

In some embodiments of the engineered nucleotide sequence disclosed herein, the desired target gene may be selected from the group consisting of, for example, but not limited to, CD25, CHD2, CFTR, CHD8, COL1A1, COL1A2, FBN1, FIGLA, FOXG1, FOXP2, FZD4, GATA2, GATA4, GI12, GLI3, HNF1B, HNF4A, HOXD13, IKZF1, IL2RA, IRF6, KIF1B, KIF21A, MECP2, MED13L, MEF2C, MEIS2, NHF1B, NKX2-5, NSD1, NR2F2, NR5A1, PAX2, PAX3, PAX5, PAX6, PBX1, PITX2, PPARG, POU3F2, RAI1, RUNX1, RUNX2, SCN1A, SETBP1, SETD1A, SIM, SIX3, SLC52A2, SOX2, SOX9, SOX10, TBX1, TBX5, TBR1, ZIC2, and ZFPM2.

In some embodiments of the engineered nucleotide sequence disclosed herein, the targeting sequences are complementary to a target sequence in a desired target gene, and the desired target gene is involved or implicated in cancer.

In some embodiments of the engineered nucleotide sequence disclosed herein, the desired target gene may be selected from the group consisting of, for example, but not limited to, AKT1, ALK, APC, ATM, BAG1, BARD1, BMPR1A, BRAF, BRCA1, BRCA2, BRIP1, CDK4, CDH1, CDK4, CDKN1C, CDKN2A, CHEK2, CHK2, CREBBP, CTNNB1, EGFR, EP300, EPCAM, ERBB2, ETV6, FBXW7, FGFR2, FHIT, FLT3, FOXL2, GNAQ, GNAS, HOXB13, HRAS, KIT, KRAS, MAP2K1, MET, MLH1, MLL, MSH2, MSH6, MYC, NF1, NTR3, NTRK1, NRAS, PALB2, PAX8, PDGFRA, PIK3CA, PMS2, PPARγ, PRCC, PRKAR1A, PTEN, RAD51D, RET, RUNXBP2, SMAD4, SRC, STK11, TFE3, TGF-β, TGF-βRII, TP53, VEGFA, and WWOX.

In another aspect, the disclosure provides a vector comprising the engineered nucleotide sequence disclosed herein.

In certain embodiments of the vector disclosed herein, the vector is a virus, a lentivirus, an adenovirus, a retrovirus, an adeno-associated virus (AAV), a transposon, a DNA vector, or a RNA vector.

In another aspect, the disclosure provides a pharmaceutical composition comprising the engineered nucleotide sequence disclosed herein, or the vector disclosed herein, and a pharmaceutically acceptable excipient. In some embodiments, the engineered nucleotide sequence disclosed herein can be administered via a pharmaceutical composition comprising the engineered nucleotide sequence disclosed herein in a lipid nanoparticle composition.

In another aspect, the disclosure provides a method for directing acetylation to a target sequence, wherein the method comprises administering the engineered nucleotide sequence disclosed herein, the vector disclosed herein, or the pharmaceutical composition disclosed herein.

In another aspect, the disclosure provides a method for treating a disease or disorder, wherein the method comprises administering the engineered nucleotide sequence disclosed herein, the vector disclosed herein, or the pharmaceutical composition disclosed herein to a subject in need thereof.

In certain embodiments of the methods disclosed herein, the disease or disorder comprises any disease or disorder where moderate protein expression can lead to alleviation of the disease or disorder phenotype (i.e., a disease or disorder with an associated haploinsufficiency).

In certain embodiments of the methods disclosed herein, the disease or disorder comprises a cancer.

In another aspect, the disclosure provides a use of the engineered nucleotide sequence disclosed herein, the vector disclosed herein, or the pharmaceutical composition disclosed herein in the treatment of a disease or disorder in a subject in need thereof.

In certain embodiments of the use disclosed herein, the disease or disorder comprises any disease or disorder where moderate protein expression can lead to alleviation of the disease or disorder phenotype (i.e., a disease or disorder with an associated haploinsufficiency).

In certain embodiments of the methods and use disclosed herein, the disease or disorder is associated with a haploinsuffciency and may be selected from the group consisting of, for example, but not limited to, Rett syndrome, Marfan syndrome, Dravet syndrome, and cancers (for example, but not limited to, PTEN or p53 null).

In certain embodiments of the methods and use disclosed herein, the method and/or use results in increased protein levels of the protein encoded by the target sequence.

In certain embodiments of the methods and use disclosed herein, the method and/or use results in decreased protein levels of the protein encoded by the target sequence.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic of the unnatural and engineered snoRNA, SAS-49, (5′-GUCCGACCUUCACCUUCCGAGCGUGAUGAUUGGGUGUUCAUACGCUUGUGUGAGAUGU GCCACCCUUUGUCUGAGCGAUGUGGUGGGCACAUUACCCGUCUGACC-3′; SEQ ID NO:9) designed to target the Kozak sequence of GAPDH mRNA (5′ CACAUCGCUCAGACACCAUGGGGAAGGUGAAGGUCGGA-3′; SEQ ID NO:10). The box indicates the putative acetylation site.

FIG. 2 shows a schematic of an excerpt of the pCMV β-globin plasmid used for the delivery of the synthetic snoRNA analogue of SNORD13 (U13). Exons 1-3, as well as the intervening introns sequences stem from the natural β-globin gene and enable transfer and expression of unnatural snoRNAs in transfected cells. The snoRNA is inserted into the intron through utilization of the restriction enzymes ClaI and XhoI.

FIG. 3 shows expression of SAS-49 increases N4-acetylcytidine levels in GAPDH mRNA. Quantitative data for N4-acetylcytidine levels in isolated GAPDH mRNA determined by UHPLC-MS/MS analysis. The control sample represents results from HeLa WT cells transfected with the empty pCMV β-globin plasmid. SAS-49 represents HeLa WT cells transfected with the pCMV β-globin plasmid containing the optimal SNORD13 analogue, SAS-49, targeting the Kozak sequence of GAPDH mRNA.

FIGS. 4A and 4B show a schematic of the NAT10/snoRNA scaffold (FIG. 4A) and positional information derived from CLIP and CLASH-seq (FIG. 4B)

FIG. 5 shows a schematic for NAT10 adapters for mRNA acetylation.

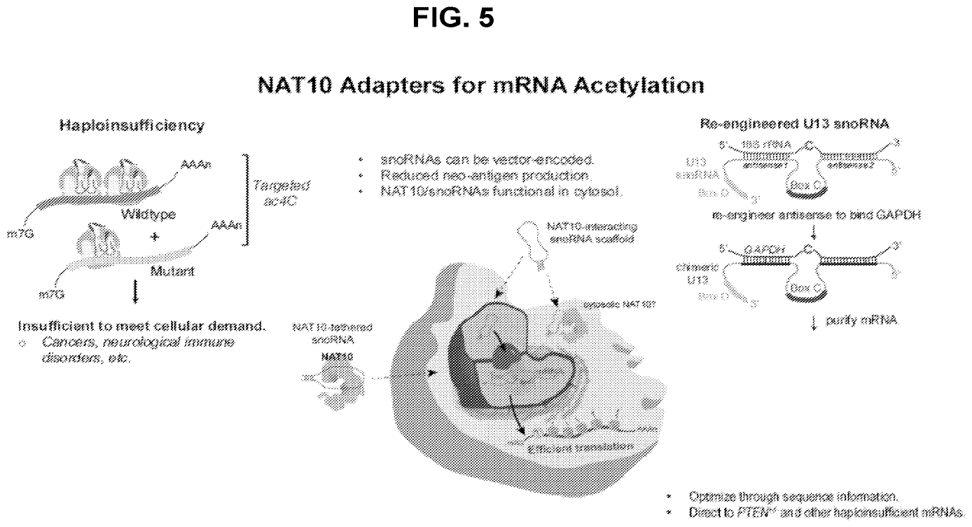

FIG. 6 shows that snoRNAs are a preferred substrate for NAT10 and establish their utility as flexible adapters. NAT10 CLIP-seq revealed 115 interactions with snoRNAs, of which 108 are of the Box C/D variety.

FIG. 7 shows that depletion of NAT10 associated SNORD14B (U14B) in HeLa cells results in decreased RNA acetylation. NAT10 CLIP-seq identified strong binding to U14B. To assess its involvement in RNA acetylation, HeLa cells were transfected with 5 individual antisense oligonucleotides (ASOs) directed against U14B. Total RNA was isolated to assess the efficiency of knockdown through RTqPCR (top) and ac4C levels through immunoNorthern (bottom). A decrease in ac4C levels was observed that correlated with knockdown efficiency.

FIG. 8 shows that chimeric U13 reproducibly increases GAPDH mRNA acetylation. Mass spectrometric quantification of ac4C levels normalized against cytidine after targeting of GAPDH mRNA with chimeric SNORD13 in wildtype HeLa cells.

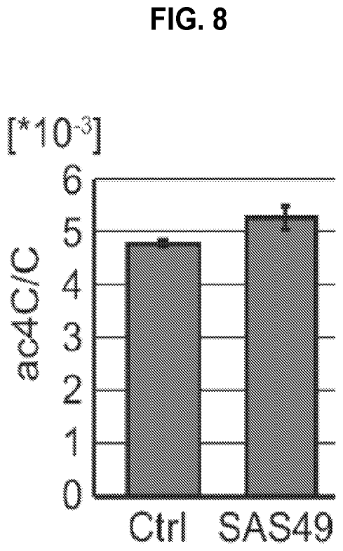

FIGS. 9A and 9B show the haploinsufficient mRNA CD25 exhibits enhanced sensitivity to ac4C as compared to PTEN mRNA. (FIG. 9A) Western blot for PTEN with GAPDH as loading control upon transfection of in vitro transcribed PTEN mRNA with varying levels of ac4C incorporation into wildtype HeLa cells. (FIG. 9B) Western blot for CD25 with GAPDH as loading control upon transfection of in vitro transcribed CD25 mRNA+/−ac4C into wild-type HeLa cells.

FIGS. 10A and 10B show that ac4C stimulates mRNA translation as compared to unmodified mRNA and mRNA containing the uridine modification pseudouridine. (FIG. 10A) A Western blot for Nanoluciferase (Nanoluc) protein levels in HeLa cells transfected with in vitro transcribed Nanoluc mRNA with either canonical nucleotides, or nucleoside triphosphates (NTPs), in which ac4C or pseudouridine (PseudoU) have been substituted for C or U, respectively. Endogenous GAPDH serves as a loading control. (FIG. 10B) Luminescence for Nanoluc in transfected HeLa cells with in vitro transcribed Nanoluc mRNA with either canonical nucleotides, or mixtures containing ac4C or PseudoU as in FIG. 10A.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE DISCLOSURE

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

Before describing the present invention in detail, a number of terms will be defined. Unless otherwise required by context, singular terms shall include pluralities and plural terms shall include the singular. For example, the singular forms “a,” “an,” and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The terms “comprises” and variations thereof do not have a limiting meaning where these terms appear in the description and claims.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the term “about” is used to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Ranges and amounts can be expressed as “about” a particular value or range. About can also include the exact amount. Typically, the term “about” includes an amount that would be expected to be within experimental error. The term “about” includes values that are within 10% less to 10% greater of the value provided.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, the terms “polynucleotide,” “nucleotide,” “oligonucleotide,” and “nucleic acid” can be used interchangeably to refer to nucleic acid comprising DNA, cDNA, RNA, mRNA, derivatives thereof, or combinations thereof.

As utilized in accordance with the present disclosure, unless otherwise indicated, all technical and scientific terms shall be understood to have the same meaning as commonly understood by one of ordinary skill in the art.

Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA). Standard recombinant DNA methodologies are used to construct the polynucleotides of the disclosure, incorporate such polynucleotides into recombinant expression vectors, and introduce such vectors into host cells to produce the amino acid sequences, proteins, and polypeptides of the disclosure. See e.g., Sambrook et al., 2001, MOLECULAR CLONING: A LABORATORY MANUAL (Cold Spring Harbor Laboratory Press, 3rd ed.). Unless specific definitions are provided, the nomenclature utilized in connection with, and the laboratory procedures and techniques of, analytical chemistry, synthetic organic chemistry, and medicinal and pharmaceutical chemistry described herein are those well-known and commonly used in the art. Similarly, conventional techniques may be used for chemical syntheses, chemical analyses, pharmaceutical preparation, formulation, delivery, and treatment of patients.

The inventors engineered chimeric snoRNA guides to recruit NAT10 to a target transcript that causes directed acetylation of the target transcript, which can regulate protein production. The inventors aim to target acetylation to specific mRNAs in cells to regulate protein synthesis (e.g., enhanced expression of tumor suppressor genes or reduced expression of oncogenes). Through transcriptome-wide analysis of RNAs that interact with the NAT10 enzyme, the inventors identified 97 novel interactions with small nucleolar RNAs (snoRNAs) that showed stronger interaction with NAT10 as compared to the previously established association with SNORD13. NAT10 is the sole enzyme to mediate acetylation in human RNA and snoRNAs are established guides for RNA-modifying enzymes. The inventors aim to determine the potential of these snoRNA to target a transcript of interest for acetylation in in vivo experiments. Endogenous NAT10 would subsequently be recruited to the targeted transcript and produce the acetylated cytidine. Mass spectrometric analysis of the transcript, and (semi-)quantitative protein expression will be used to validate formation of ac4C and effects on protein biosynthesis. As proof of principle, the inventors modulated the NAT10-interacting snoRNA SNORD13 to target GAPDH mRNA.

The engineered nucleotides (e.g., snoRNAs) as disclosed herein comprise (5′ to 3′) a first targeting sequence, a C box motif, a second targeting sequence, and a D box motif. The first targeting sequence can be from about 15 to about 30 nucleotides in length, for example 18-28 nucleotides, or 21-25 nucleotides. The C box motif can be about 40 to about 200 nucleotides in length, and comprises the sequence 5′ RUGAUGA-3′ (wherein R is a purine) or a portion thereof. The second targeting sequence can be from about 15 to about 30 nucleotides in length, for example 18-28 nucleotides, or 21-25 nucleotides. The D box motif can be about 15 to about 50 nucleotides in length (e.g., and can be substantially complementary to a portion of a target sequence). In some embodiments, the D box motif comprises a 5′-CUGA-3′ sequence.

In certain embodiments, first targeting sequence and the second targeting sequence are substantially complementary to separate regions of a target sequence. In certain embodiments, first targeting sequence and the second targeting sequence are substantially complementary to separate regions of a target sequence in the same desired target gene. A target sequence serves to hybridize to a target nucleic acid sequence (i.e., the mRNA of a desired target gene for which modified expression is the result). Typically the first targeting sequence and the second targeting sequence are substantially complementary to a portion of target nucleic acid sequence that is about is 15-30 nucleotides in length, but may be longer depending upon sequence and base composition. The term “substantially” complementary means that there does not need to be exact complementarity between the target nucleic acid sequence and first targeting sequence and the second targeting sequence of the engineered nucleotide sequence. However, it is to be understood that there should be a high degree of sequence identity, typically greater than 90% or 95%, which is sufficient to ensure specific binding according to known RNA or DNA/RNA base pairing rules. As used herein, the term “hybridize” refers to the sequence specific non-covalent binding interaction with a complementary nucleic acid. In one embodiment, the hybridization is under high stringency conditions. Appropriate stringency conditions that promote hybridization are known to those skilled in the art.

In certain embodiments, the desired target gene can be a gene involved or implicated in any disease or disorder where moderate protein expression can lead to alleviation of the disease or disorder phenotype (i.e., a disease or disorder with an associated haploinsufficiency). In certain embodiments, the disease or disorder is associated with a haploinsuffciency and may be selected from the group consisting of, for example, but not limited to, Holt-Oram syndrome, Rett syndrome, Marfan syndrome, Dravet syndrome, Smith-Magenis syndrome, Waardenburg syndrome, Williams syndrome, and cancers (for example, but not limited to, PTEN or p53 null). In some embodiments, the desired target gene encodes a ribosomal protein, or the desired target gene encodes a gene involved in RNA processing, or a gene involved in gene transcription and/or translation, or a gene involved in proteostasis. For example, the desired target gene can be one or more of, but not limited to, CD25, CHD2, CFTR, CHD8, COL1A1, COL1A2, FBN1, FIGLA, FOXG1, FOXP2, FZD4, GATA2, GATA4, GI12, GI13, HNF1B, HNF4A, HOXD13, IKZF1, IL2RA, IRF6, KIF1B, KIF21A, MECP2, MED13L, MEF2C, MEIS2, NHF1B, NKX2-5, NSD1, NR2F2, NR5A1, PAX2, PAX3, PAX5, PAX6, PBX1, PITX2, PPARG, POU3F2, RAI1, RUNX1, RUNX2, SCN1A, SETBP1, SETD1A, SIM, SIX3, SLC52A2, SOX2, SOX9, SOX10, TBX1, TBX5, TBR1, ZIC2, and ZFPM2.

In certain embodiments, the desired target gene can be a gene involved or implicated in cancer. For example, the desired target gene can be one or more of, but not limited to, AKT1, ALK, APC, ATM, BAG1, BARD1, BMPR1A, BRAF, BRCA1, BRCA2, BRIP1, CDK4, CDH1, CDK4, CDKN1C, CDKN2A, CHEK2, CHK2, CREBBP, CTNNB1, EGFR, EP300, EPCAM, ERBB2, ETV6, FBXW7, FGFR2, FHIT, FLT3, FOXL2, GNAQ, GNAS, HOXB13, HRAS, KIT, KRAS, MAP2K1, MET, MLH1, MLL, MSH2, MSH6, MYC, NF1, NTR3, NTRK1, NRAS, PALB2, PAX8, PDGFRA, PIK3CA, PMS2, PPARγ, PRCC, PRKAR1A, PTEN, RAD51D, RET, RUNXBP2, SMAD4, SRC, STK11, TFE3, TGF-β, TGF-βRII, TP53, VEGFA, and WWOX.

In some embodiments, the target sequence is an RNA sequence, typically an mRNA, tRNA, miRNA or rRNA sequence, or an RNA virus genomic sequence or transcript derived thereof of the desired target gene. In certain embodiments, the target sequence is an mRNA sequence. In certain embodiments, the target sequence is a coding sequence (CDS) within the mRNA sequence. Within a target nucleic acid sequence the complementary region can be either in the intron, or exon, or particularly at intron-exon junctions, but can also reside in 5′ or 3′ untranslated regions. In certain embodiments, targeting of the engineered nucleic acids as disclosed herein to the CDS of a desired target gene can increase protein production of the desired target gene.

In certain embodiments, the target sequence is in the 5′ untranslated region of an mRNA transcript. In certain embodiments, the target sequence is in the 3′ untranslated region of an mRNA transcript. In certain embodiments, targeting of the engineered nucleic acids as disclosed herein to the 5′ untranslated region of a desired target gene can repress protein production of the desired target gene.

In some embodiments, the target sequence comprises a known acetylation site or a putative acetylation site. In some embodiments, the target sequence is within about 30 to about 50 nucleotides of a known acetylation site or a putative acetylation site.

In a further aspect, this disclosure provides a vector or other nucleic acid construct capable of expressing at least one engineered nucleotide sequence as disclosed herein. The vector may further comprise common nucleic acid features often found in conventional nucleic acid vectors and well known to the skilled artisan. For example, the vector may comprise a selection marker gene for facilitating identification of cells into which the nucleic acid construct has been transformed or transfected. In some embodiments, the vector comprises at least one promoter, such as a constitutive or controllable promoter known in the art, for facilitating expression of the nucleic acid encoding engineered nucleotide sequences disclosed herein. In certain embodiments, the vectors as disclosed herein may also be delivered into a cell using viral vectors, such as lentivirus, retrovirus or adenovirus-derived vectors known in the art.

In a further aspect, this disclosure provides pharmaceutical compositions comprising the engineered nucleotides as disclosed herein, or the vectors capable of the engineered nucleotides as disclosed herein and a pharmaceutically acceptable carrier therefore.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

This disclosure also provides methods of directing acetylation to a target sequence of a desired target gene, which can regulate gene expression of the desired target gene. Thus, the engineered nucleotide sequence as disclosed herein can be used to treat a disease or condition in a subject where it is desirable to modulate (either up-regulate or down-regulate) expression of a desired target gene or genes. For example, acetylation of the CDS of a desired target gene can increase protein production (in particular, beneficial for haploinsufficiency disorders). For example, targeting to a 5′-UTR of a desired target gene can be stimulatory in upstream/alternative CUG initiation sites (for example, stimulate anti-oncogenic protein isoforms) and repressive in annotated AUG initiation sites or surrounding Kozak sequence (for example, potential target to suppress oncogenes in cancer).

This disclosure also provides methods comprising the step of administering to a subject in need thereof, a therapeutically effective amount of the engineered nucleotide sequence, the vector, or the pharmaceutical composition as disclosed herein to a subject in need thereof.

As used herein the phrase “treating” refers to inhibiting or arresting the development of a disease, disorder or condition and/or causing the reduction, remission, or regression of a disease, disorder or condition in an individual suffering from, or diagnosed with, the disease, disorder or condition. In some embodiments, the disease or disorder is associated with a haploinsuffciency. In some embodiments, the disease or disorder is selected from the group consisting of, for example, but not limited to Rett syndrome, Marfan syndrome, and Dravet syndrome. In some embodiments, the disease or disorder is cancer. In certain embodiments, targeting of the engineered nucleic acids as disclosed herein to a CDS of a desired target gene can increase protein production of the desired target gene. In certain embodiments, targeting of the engineered nucleic acids as disclosed herein to a 5′ untranslated region of a desired target gene can repress protein production of the desired target gene.

Without limiting the disclosure, a number of embodiments of the disclosure are described below for purpose of illustration.

The invention will be further described in the following examples, which do not limit the scope of the invention described in the claims.

### Examples

The Examples that follow are illustrative of specific embodiments of the disclosure, and various uses thereof. They are set forth for explanatory purposes only, and should not be construed as limiting the scope of the invention in any way.

Previous results have identified a dual role of ac4C in mRNA translation elongation and initiation (1, 2). In elongation, acetylation within coding sequences (CDS) enhances mRNA stability and protein translation (2). In initiation, acetylation of Kozak sequences reduced translation, whereas ac4C in upstream translation initiation (upTIS) CUG sites enhanced alternative translation initiation. Provided herein is an engineered chimeric snoRNA composed of the Box C/D snoRNA SNORD13 and antisense elements directed to, as proof of principle, GAPDH mRNA. The engineered snoRNA was delivered into human cells through incorporation into a minigene vector. Recovery of GAPDH transcripts and mass spectrometry confirmed successful targeted acetylation in vitro.

Engineering for the Targeting of the Kozak Region of GAPDH mRNA

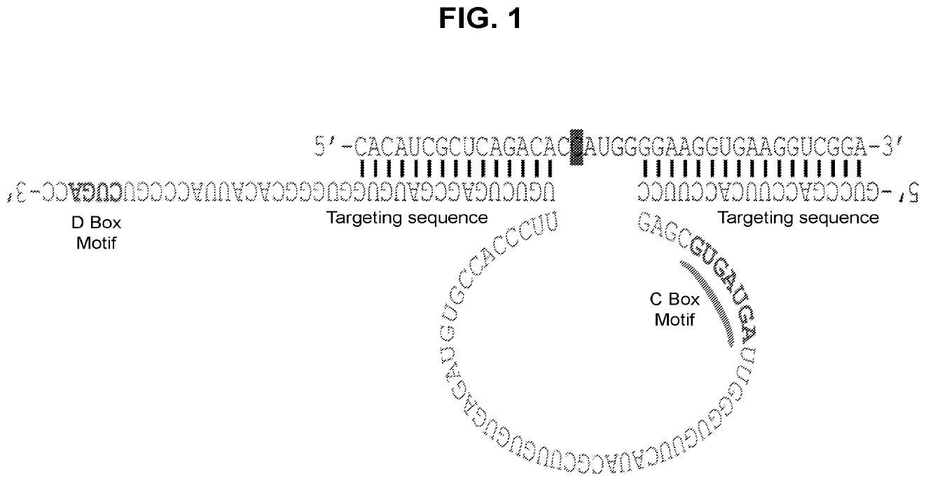

SNORD13 was identified to exhibit two imperfect targeting sequences surrounding the target cytidine in helix 45 of 18S rRNA. The inventors designed a homologue of this snoRNA (FIG. 1) with two targeting sequences around the Kozak cytidine that match the GAPDH mRNA sequence to 100%. (Sequence SAS-49):

(SEQ ID NO: 1)
5′ GTCCGACCTTCACCTTCCGAGCGTGATGATTGGGTGTTCATACGCTT
GTGTGAGATGTGCCACCCTTTGTCTGAGCGATGTGGTGGGCACATTACCC
GTCTGACC 3′

Expression of Targeting snoRNAs

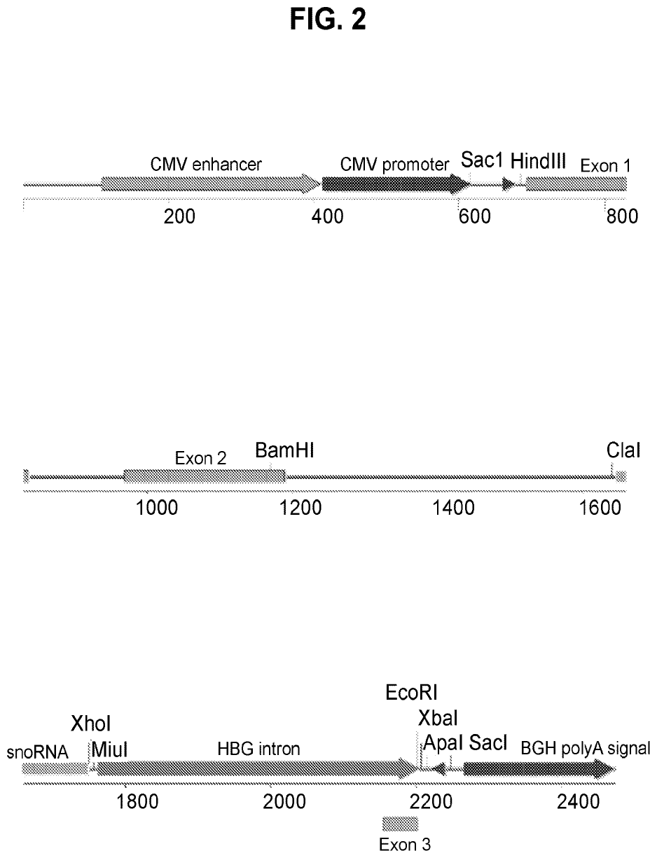

In previous studies (3) it was shown that snoRNAs inserted into an intron of the β-globin gene can be processed to mature, functional snoRNAs. Kiss et al. developed a plasmid (FIG. 2) containing introns and exons of the β-globin gene with restriction sites for insertion of any kind of snoRNA.

Despite the fact that the processing of SNORD13 in nature occurs differently because it is regulated and transcribed from an independent gene (4), the inventors hypothesize that a synthetic snoRNA might be expressed in vivo via the same pathway as snoRNAs that are naturally processed from introns.

Engineering of pCMV β-Globin Plasmid Containing the Synthetic snoRNA

The plasmid provided by the Filipowicz lab (3) was digested using the ClaI and XhoI restriction enzymes and the ends were dephosphorylated using the phosphatase rSAP. The snoRNA analogue was purchased from IDT with the respective restriction sites on the 5′ and 3′ end and digested to yield compatible ends. Both fragments were purified via column purification (PCR Clean-Up kit, Qiagen). Utilizing T4 DNA ligase, the snoRNA was inserted into the plasmid overnight at 16 °C and the product transformed into DH5α cells. Colony-PCR with T7 FWD and SAS-51/BGH polyA REV primers revealed good candidates which were confirmed by Sanger sequencing. The material was amplified through a standard midi-prep protocol (ZymoResearch).

Transfection into HeLa WT Cells

To study the expression of the synthetic SNORD13 derivative that targets the Kozak sequence of GAPDH mRNA, a plasmid containing the @-globin gene with introns harboring the snoRNA sequence was transfected. To this end HeLa WT cells cultured routinely in DMEM medium supplemented with 1% L-glutamine and 10% bovine calf serum were seeded to 2.2×10⁶ cells into a 10 cm culture dish and allowed to attach at 37 °C in atmosphere containing 5% CO₂. After 24 h the plasmid DNA was transfected according to the standard Lipofectamine 2000 (Invitrogen) protocol using 10 μg of plasmid DNA and 60 μL of Lipofectamine 2000 solution. The medium was replaced with fresh medium after 4-5 h and the cells were cultured for a total of 24 h after transfection.

Isolation of GAPDH mRNA after Transfection of the pCMV β-Globin Plasmid Containing the Synthetic snoRNA

The cells were harvested for RNA after 24 h by scraping with 1 mL TRIZOL (Invitrogen). The total RNA was isolated according to the standard protocol. From total RNA the GAPDH mRNA was enriched utilizing a biotinylated antisense oligo (biotin-ASO) according to a previously published protocol (5). In brief, the RNA (up to 150 μg) was diluted in 50 μL ultrapure water. The biotin-ASO complementary to the GAPDH Kozak sequence (SAS-41, 1 μL, 100 μmol) was added and the volume brought to 100 μL with ultrapure water and 25 μL 20×SSC buffer. Magnetically separated DynaBeads My One Streptavidin-T1 (25 μL) were washed three times with buffer A (1 M NaCl, 0.5 mM Na₂[EDTA], 5 mM TRIS-HCl, pH 7.5), once with 5×SSC buffer (25 μL) before resuspension in 5×SSC buffer (25 μL). The RNA was heated for 3 min at 90 °C, then at 65 °C for 10 min and finally allowed to cool to room temperature. The prepared beads (25 μL) were added to the sample and incubated for 30 min at 600 rpm and 25 °C. Subsequently, the beads were washed once with 1×SSC buffer (50 μL) and three times with 0.1×SSC buffer (50 μL). Finally, the beads were resuspended in ultrapure water (15 μL), incubated at 75 °C for 2 min and the RNA transferred to a new tube.

HPLC-MS/MS Analysis of GAPDH mRNA

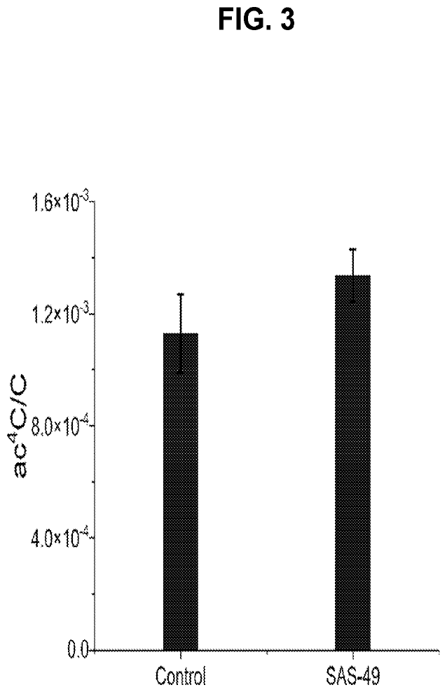

As quality check, enriched GAPDH mRNA was subjected to mass spectrometric analysis. To this end, enriched GAPDH mRNA (100 ng) was diluted to a total volume of 20 μL with nuclease-free water. A master mix of snake venom phosphodiesterase (0.2 U), calf intestinal phosphatase (2 U) and benzonase (2 U) was prepared with 1 mM MgCl₂, 5 mM TRIS (pH 8), 10 nmol butylated hydroxytoluene and 5 g tetrahydrouridine (modified from Heiss (2021); (5)) containing isotope analogues of N⁶-methyladenosine, 5-Methylcytidine and N⁴-acetylcytidine as internal standards. After addition of 15 μL of the mastermix, samples were incubated at 37 °C for 2 h. The samples were diluted with 15 μL LC-MS buffer A (0.0075% formic acid in ultrapure water) and filtered for 35 min at 3750 g and 4 C through 0.2 μm Supor AcroPrep Advance 96-well plates (Pall corporation). Of the filtrate, 39 μL were subjected to mass spectrometric analysis on an Agilent triple quad 6495C mass spectrometer. For HPLC, buffer A (described above) and buffer B (0.0075% formic acid in acetonitrile) were used in combination with a C-8 reversed phase column (Agilent Poroshell 120 SB-C8, 2.1×150 mm, 2.7 um LC column—BC). The gradient settings and nucleoside-specific parameters in the mass spectrometer (MS) are detailed in Table 2 and 3. The parameters of the MS were the following: 35 °C column temperature, 90 °C gas temperature, 19 L/min gas flow, 166 V fragmentor voltage, 30 psi nebulizer, 150 °C sheath gas temperature, 12 L/min sheath gas flow, 0 V nozzle voltage, positive high pressure RF 80, positive low pressure RF 40, negative high pressure RF 80, negative low pressure RF 40, 2500 V and −2000 V capillary voltage, respectively. All data were analyzed according to a published protocol utilizing the isotope dilution technique (6). Data (FIG. 3) confirm an increase of acetylation in GAPDH mRNA after expression of an engineered snoRNA based on SNORD13 (SAS-49). A chimeric U13 reproducibly increased GAPDH mRNA acetylation (based on mass spectrometric quantification of ac4C levels normalized against cytidine after targeting of GAPDH mRNA with chimeric SNORD13 in wild-type HeLa cells).

### Validation Controls

Various controls will ensure integrity of the data. To rule out transfection artifacts non-transfected cells serve as negative control. The effect of the addition of the β-globin plasmid will be evaluated by transfecting the empty pCMV β-globin plasmid. As non-targeting control the inventors seek to use the natural SNORD13 sequence. Due to its natural processing pathway and abundance (~2×10⁵ copies/cell in HeLa; (7)) in the cells, the inventors do not expect major effects, but this control is important to assess the effect of aberrant SNORD13 processing.

snoRNAs for Directed mRNA Acetylation in Mammalian Subjects

The snoRNA guide disclosed herein demonstrates proof of principle in cell lines through transfection of a DNA-vector. For delivery into patients, methods could include the use of Adeno-associated virus (AAV) or lentiviral vectors. Such vectors are ideally suited for the delivery of the snoRNA guides. AAV and lentiviral vectors are already being used/investigated clinically, so application of this therapy using such vectors appears feasible.

Further, these snoRNA guides and methods of delivery as disclosed herein can be developed as a patient-specific treatment, as well as a generic treatment. In vivo studies are performed to identify whether protein expression of a particular haploinsufficiency gene(s) increases upon acetylation. Subsequently, the optimized snoRNA guide are identified and engineered for those specific haploinsufficiency gene(s). Once the guide is identified, the vector-encoded snoRNA could be made broadly available to any patient suffering from that specific haploinsufficiency.

snoRNAs for Guided mRNA Acetylation

NAT10 activity can be manipulated to develop novel therapeutics for specific defects in mRNA translation. Traditional gene therapy approaches rely on eliminating deleterious transcripts. However, strategies that involve genome manipulation carry inherent risks, and mRNA ablation does not address the numerous human diseases that result for inadequate mRNA translation, such as haploinsufficiency disorders (8). Haploinsufficiency is a genetic condition that arises when a single wild-type allele in combination with a mutant allele produces insufficient protein, manifesting in disease (9). There are over 300 known, and perhaps thousands of additional, human haploinsufficiencies (10, 11). This class of disease is generally recalcitrant to therapeutic interventions as technologies for enhancing the abundance of specific proteins are lacking. In addition, many haploinsufficiencies arise from spontaneous germline mutations (12), rendering gene editing approaches untenable. Recent RNA-based therapeutics have aimed to address this unmet need through direct delivery of codon optimized mRNAs and through ASO technologies that promote protein expression via exclusion of toxic-exons (13). Though promising in principle, mRNA delivery is size-constrained and involves uniform incorporation of modified RNA nucleotides that may exert antagonistic influences and ASO technologies are limited to pre-mRNAs with toxic exons (14). Provided herein are engineered snoRNA guides for targeting endogenous NAT10 to specific mRNA substrates in vivo as a versatile and mutation-agnostic platform for fine-tuning protein expression at the level of mRNA translation.

snoRNA Guides

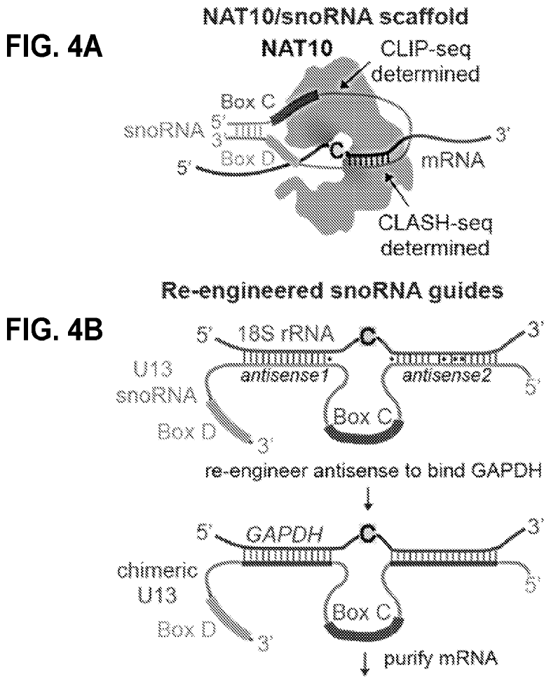

The successful development of these engineered snoRNA guides hinges on the ability to bind NAT10 and mRNA in cells. As proof-of-concept, the inventors re-engineered U13 snoRNA to target GAPDH mRNA proximal to its start codon, rather than 18S rRNA, through mutating the two antisense guide elements (FIG. 4). Chimeric U13 was cloned into the beta-globin minigene and transfected into HeLa cells. GAPDH mRNA was recovered from cell lysates through biotinylated antisense probe and subjected to mass spectrometric analysis for ac4C prevalence. Preliminary quantification detected a ~20% increase in ac4C within chimeric U13 targeted GAPDH mRNA as compared to control transfected cells (FIG. 3). Considering that haploinsufficiencies can display dosage sensitivity, wherein small increases in protein expression are beneficial, while large increases are detrimental (15), this initial value is significant. Importantly, ac4C influenced total protein abundance within this stoichiometry range in HeLa mRNAs (1). To next gauge specificity with respect to precisely acetylated residues RetraC:T and nanopore sequencing is performed on the recovered transcripts. The overall impact on GAPDH expression in cell lysates is also examined. Considering that NAT10 binding to U13 was relatively reduced as compared to other snoRNAs in HeLa CLIP-seq, these promising preliminary findings bolster the development of improved snoRNA scaffolds.

To design optimal NAT10-binding snoRNAs, HeLa NAT10 eCLIP-seq and CLASH-seq results were interrogated for generalized principles. In brief, the presence of abrupt RT-stops in eCLIP enriched sequencing reads are often indicative of direct RBP binding. Homology search of NAT10-associated snoRNAs revealed several conserved sequences that generally overlapped eCLIP-seq RT-stops, strongly suggestive that the associated sequences are involved in NAT10-binding. These regions formed the basis of an optimized NAT10 scaffold along with consensus Box C and Box D motifs several nucleotides from the 5′ and 3′ ends, respectively (FIG. 4A). The remainder of the scaffold and specific antisense placement was guided by CLASH-seq determination of substrate-interacting regions. Antisense targeting sequences were directed to GAPDH as proof of principle and other abundant mRNAs (e.g., CD25 and PTEN) to confirm acetylation through mass spectrometry and base-resolution RNA-sequencing. The net impact on protein expression as compared to non-target mRNAs was also examined. The scaffolds were optimized through sequence randomization. In parallel, optimized U13 snoRNA scaffolds were pursued through mutagenesis. To evaluate specificity, a pool of the top candidates was expressed in HeLa and NAT10 eCLIP/CLASH-seq was performed to verify NAT10/snoRNA association and assess interacting mRNAs. The engineered snoRNA guides were tested by mass spectrometric analysis for inadvertent targeting of 2′-O-methylation to non-substrates. This disclosure provides a reliable snoRNA scaffold to target NAT10 to specific mRNAs through a flexible antisense platform.

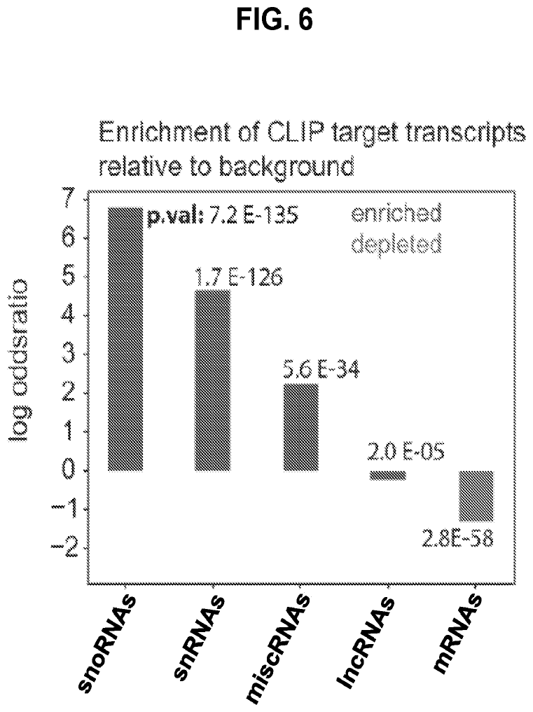

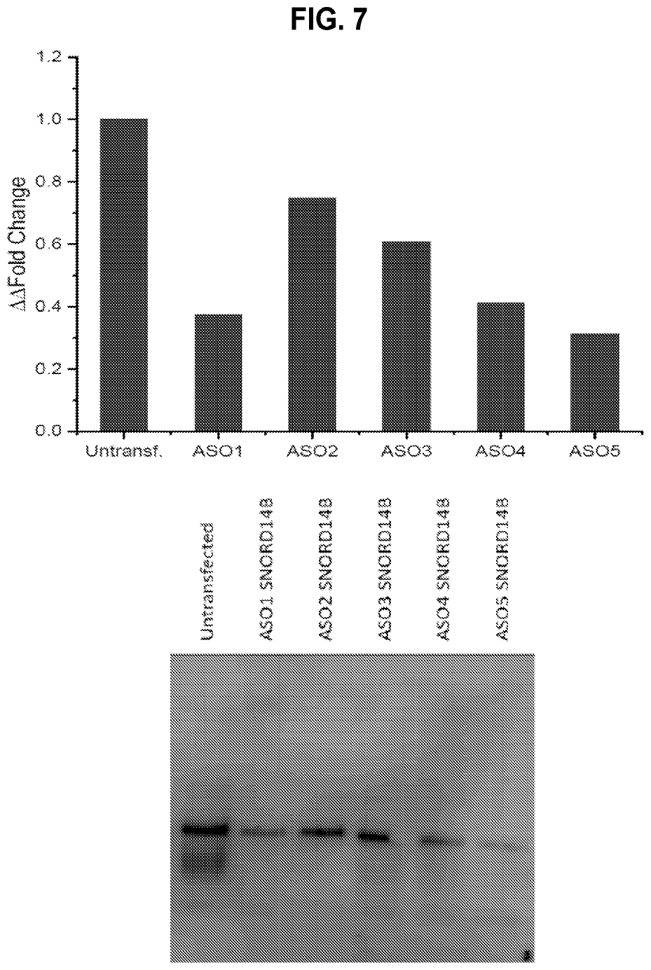

NAT10 CLIP-seq was performed and revealed 2460 CLIP sites that are enriched in wild-type HeLa as compared to control. Examination of the substrate biotypes discovered a strong prevalence of snoRNAs demonstrating that NAT10-CLIP targets are enriched for snoRNAs (FIG. 6). Furthermore, depletion of NAT10 associated SNORD14B in HeLa cells resulted in decreased RNA acetylation. HeLa cells were transfected with 5 individual ASOs directed against U14B. Total RNA was isolated to assess the efficiency of knockdown through RTqPCR and ac4C levels through immunoNorthern. A decrease in ac4C levels was observed that correlated with knockdown efficiency (FIG. 7).

### Knockdown of SNORD14B—Cell Culture

HeLa WT cells maintained in complete (containing 1% L-glutamine and 10% bovine calf serum (BCS)) Dulbecco's modified Eagle's medium (DMEM) at 37 °C in atmosphere containing 5% CO₂ were trypsinized and counted. For each condition, 200,000 cells were seeded into 6 wells. The medium was completely replaced with OptiMEM 24 hours after seeding, and for each condition 50 nM antisense-oligo (ASO1-5, Table 1) was transfected using Lipofectamine 2000 reagent in a total volume of 200 μL according to manufacturer's instructions. The medium was replaced with fresh complete DMEM after 4 hours. The cells were lysed directly in 500 μL TRIZOL reagent (Invitrogen) 48 hours after transfection.

TABLE 1
Antisense-oligos utilized in SNORD14B knockdown experiments.
PO = phosphate backbone,
2′-MOE = 2′-O-methoxyethyl-modified ribose.

| | Sequence (5' -3') | Modification | Length | snoRNA |
|---|---|---|---|---|
| ASO1 | AGACATCCAAGGAAGGTTTA | All PO | 20 | SNORD14B |
| | (SEQ ID NO: 2) | 1-5 + 16-20 2′-MOE | | |
| ASO2 | ACATCCAAGGAAGGTT | All PO | 16 | SNORD14B |
| | (SEQ ID NO: 3) | 1-4 + 13-16 2′-MOE | | |
| ASO3 | TAAGGAAAACCTTTCTGGTG | All PO | 20 | SNORD14B |
| | (SEQ ID NO: 4) | 1-5 + 16-20 2′-MOE | | |
| ASO4 | TTCTGGTGGAAACTGCGAAT | All PO | 20 | SNORD14B |
| | (SEQ ID NO: 5) | 1-5 + 16-20 2′-MOE | | |
| ASO5 | CTGCGAATGTTGGAAAACCA | All PO | 20 | SNORD14B |
| | (SEQ ID NO: 6) | 1-5 + 16-20 2′-MOE | | |

### Knockdown of SNORD14B—RNA Isolation and Analysis

RNA was extracted according to manufacturer's instructions. Briefly, 2 g of RNA were diluted to a total volume of 10 μL and 2× formamide loading dye (95% formamide, 0.5 mM EDTA, traces of bromophenol blue and xylene cyanol FF) was added to a volume of 20 μL. The RNA was heated for 5 minutes at 65 °C and separated on an 1% agarose gel containing 1×MOPS (NorthernMax™ Denaturing Gel Buffer (10×), Catalog number: AM8676). Subsequently, the RNA was transferred to a HyBond nylon membrane with 20× sodium citrate/chloride buffer for 3 hours at room temperature and cross-linked twice to the membrane with the auto-crosslink function of the Stratalinker (120 mJ/cm2). After quick rinse with phosphate buffered saline (PBS), the membrane was blocked for 15 minutes in 5% milk and PBS containing 0.01% Tween-20 (PBS-T). The membrane was incubated with primary antibody against ac4C (monoclonal rabbit, 1:1000 in 1% milk/PBS-T) overnight at 4 °C. After three washes for 5 minutes with PBS-T, the membrane was incubated with secondary horseradish peroxidase (HRP) conjugated antibody against rabbit IgG (monoclonal mouse, 1:10,000 in 5% milk/PBS-T, Jackson ImmunoResearch 211-032-171). The membrane was washed three times with PBS-T, incubated for 2 minutes with Femto reagent (Thermo Fisher Scientific) and the chemiluminescent signal captured with the Biorad ChemiDoc machine.

For quantitative reverse transcription polymerase chain reaction (qRTPCR), 1 μg of RNA was converted into cDNA with superscript IV reverse transcriptase using random hexamers. The cDNA was subsequently subjected to qPCR on an LightCycler 480 Instrument (Roche Diagnostics) using the SYBRGreen 2× mastermix from Roche Diagnostics and SNORD14B-specific primers (forward: 5′-TCACTGTGATGATGGTTTTC-3′ (SEQ ID NO:7) and reverse: 5′-CTCACTCAGACATCCAAGGA-3′ (SEQ ID NO:8)).

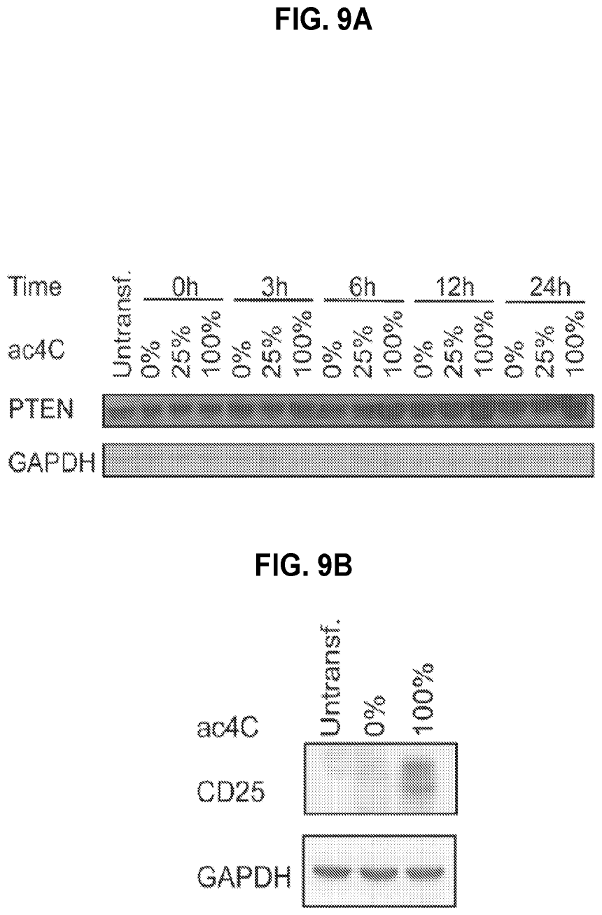

As shown in FIGS. 9A and 9B, haploinsufficient CD25 mRNA shows enhanced sensitivity to ac4C as compared to PTEN mRNA. Western blot for PTEN with GAPDH as loading control upon transfection of in vitro transcribed PTEN mRNA with varying levels of ac4C incorporation into wildtype HeLa cells. Western blot for CD25 with GAPDH as loading control upon transfection of in vitro transcribed CD25 mRNA+/−ac4C into wild-type HeLa cells.

Generation of Reporter mRNA

To compare the effect of ac4C on nanoluciferase, PTEN and CD25 expression with pseudouridine and unmodified mRNA, the transcripts were in vitro transcribed from a template containing no cytidines in the 5-untranslated region, Kozak sequence and first 15 nucleotides of the coding sequence, as well as a 20 nucleotide 3′-UTR. The acetylated and pseudouridylated mRNAs were achieved by omission or reduction of cytidine and uridine, respectively. The transcripts were simultaneously capped with AG-CleanCap (TriLink) according to manufacturer's protocol. PTEN and CD25 were subsequently polyadenylated with E. coli polyA-polymerase (according to manufacturer's protocol). The construct for nanoluciferase included templated poly(A)-sequence making polyadenylation with E. coli polyA-polymerase obsolete.

Transfection of PTEN mRNA into HeLa WT Cells

HeLa WT cells maintained in complete (containing 1% L-glutamine and 10% bovine calf serum (BCS)) Dulbecco's modified Eagle's medium (DMEM) at 37 °C in atmosphere containing 5% CO₂ were trypsinized and counted. For each condition, 200,000 cells were seeded in 12 wells in biological triplicates and grown for 8 hours at 37 °C in atmosphere containing 5% CO₂. The transfections were performed at different time points, either 24 hours, 12 hours, 6 hours, 3 hours or 0 hours before cell harvest. For each condition and time point, 333 ng of PTEN mRNA (0-100% ac4C) and 167 ng of unmodified Nanoluciferase mRNA were transfected utilizing 0.75 μL Lipofectamine MessengerMax reagent with a total volume of 30 μL OptiMEM according to manufacturer's instructions. At time point 0 hours after transfection, the cells were washed with PBS, trypsinized for 3 minutes, quenched with complete DMEM medium, washed twice with PBS and lysed in RIPA buffer containing 1% protease inhibitor.

### Western Blot to Determine PTEN Levels

The protein concentration was determined with the Pierce BCA Protein Assay (Thermo Fisher Scientific) and 25 μg separated for 45 minutes at 200 V on 4-12% BisTris gels (Invitrogen) using MOPS buffer (Invitrogen). The proteins were transferred to PVDF membranes using the Biorad TurboBlot midi gel mixed molecular weight settings for 7 minutes. The membranes were blocked with 5% milk in TRIS-buffered saline (TBS) containing 0.01% Tween-20 (TBS-T). The membrane was incubated with primary antibody against PTEN (monoclonal rabbit, 1:1000 in 5% milk/TBS-T, Cell Signaling Technologies 9559S) overnight at 4 °C on a rocking device. The following day, the membrane was washed three times for 5 minutes with TBS-T and then incubated for 45 minutes at room temperature with secondary horseradish peroxidase (HRP)-conjugated antibody against rabbit IgG (monoclonal mouse, 1:10,000 in 5% milk/TBS-T, Jackson ImmunoResearch 211-032-171). The membrane was washed three times with TBS-T, incubated for 2 minutes with ECL-Select reagent (Amersham), rinsed quickly with TBS and the chemiluminescent signal captured with the Biorad ChemiDoc machine. The membrane was rinsed again and blocked for 1 hour with 5% milk/TBS-T. The membrane was incubated with primary antibody against GAPDH (monoclonal mouse, 1:10,000 in 5% milk/TBS-T, Santa Cruz Biotechnology SC32233) overnight at 4 °C on a rocking device. The following day, the membrane was washed three times for 5 minutes with TBS-T and then incubated for 1 hour at room temperature with secondary horseradish peroxidase (HRP)-conjugated antibody against mouse IgG (polyclonal goat, 1:10,000 in 5% milk/TBS-T, abcam ab6789). The membrane was washed three times with TBS-T, incubated for 2 minutes with ECL reagent (Amersham) and the chemiluminescent signal captured with the Biorad ChemiDoc machine.

Transfection of CD25 mRNA into HeLa WT Cells

HeLa WT cells routinely maintained in complete DMEM at 37 °C in atmosphere containing 5% CO₂ were trypsinized and counted. For each condition, 1×106 cells were seeded into 6 wells the day before the transfection. For each condition, 2.5 g of mRNA utilizing 3.75 μL Lipofectamine MessengerMax reagent in a total volume of 250 μL were transfected according to manufacturer's instructions. The medium was replaced with fresh complete DMEM containing 1% L-glutamine and 10% bovine calf serum (BCS) after 4 hours. After 24 h, the cells were trypsinized, washed twice with PBS and lysed in RIPA buffer containing 1% protease inhibitor. The protein concentration was determined with the Pierce BCA Protein Assay (Thermo Fisher Scientific) and 40 μg of protein separated on 4-12% BisTris gels using MOPS buffer. The proteins were transferred to a nitrocellulose membrane using the Biorad TurboBlot mixed molecular weight settings for 7 minutes. The membranes were blocked with 5% milk in TRIS-buffered saline (TBS) containing 0.01% Tween-20 (TBS-T). The membrane was incubated with primary antibody against CD25 (monoclonal rabbit, 1:1000 in 5% milk/TBS-T, abcam ab231441) overnight at 4 °C on a rocking device. The following day, the membrane was washed three times for 5 minutes with TBS-T and then incubated for 1 hour at room temperature with secondary horseradish peroxidase (HRP)-conjugated antibody against rabbit IgG (monoclonal mouse, 1:10,000 in 5% milk/TBS-T, Jackson ImmunoResearch 211-032-171). The membrane was washed three times with TBS-T, incubated with HRP-substrate and the chemiluminescent signal captured with the Biorad ChemiDoc machine.

Transfection of Nanoluciferase mRNA into HeLa WT Cells

HeLa WT cells routinely maintained in complete (containing 1% L-glutamine and 10% bovine calf serum (BCS)) Dulbecco's modified Eagle's medium (DMEM) at 37 °C in atmosphere containing 5% CO2 were trypsinized and counted. For each condition in biological triplicates, 1×106 cells were transfected with 2.5 μg of mRNA utilizing 3.75 μL Lipofectamine MessengerMax reagent in a total volume of 250 μL (according to manufacturer's instructions). After incubation of cells with mRNA for 5 min, the medium was removed, the cells washed twice with PBS, and 10,000 cells each in triplicate were seeded into luciferase plate and at the indicated time points NanoGlo reagent (Promega) was added and the luminescence recorded with the SpectraMax iD3. The remaining cells were plated into 6 wells each. At the indicated time points, the cells were scraped, washed twice with PBS and counted. 250,000 cells were lysed in RIPA buffer containing 1% protease inhibitor. The protein concentration was determined with the BCA protein quantitation assay and 10 μg separated on 4-12% BisTris gels using MOPS buffer. Proteins were transferred to a nitrocellulose membrane using the Biorad TurboBlot mixed molecular weight settings for 7 min. The membranes were blocked with 5% milk in TRIS-buffered saline (TBS) containing 0.01% Tween-20 (TBS-T). The membrane was incubated with primary antibody against Nanoluciferase (monoclonal mouse, 1:500 in 5% milk/TBS-T, R&D Systems MAB10026) overnight at 4 °C on a rocking device. The following day, the membrane was washed three times for 5 min with TBS-T and then incubated for 1 h at room temperature with secondary secondary HRP-conjugated antibody against mouse IgG (polyclonal goat, 1:10,000 in 5% milk/TBS-T, abcam ab6789). The membrane was washed three times with TBS-T, incubated with HRP-substrate and the chemiluminescent signal captured with the Biorad ChemiDoc machine.

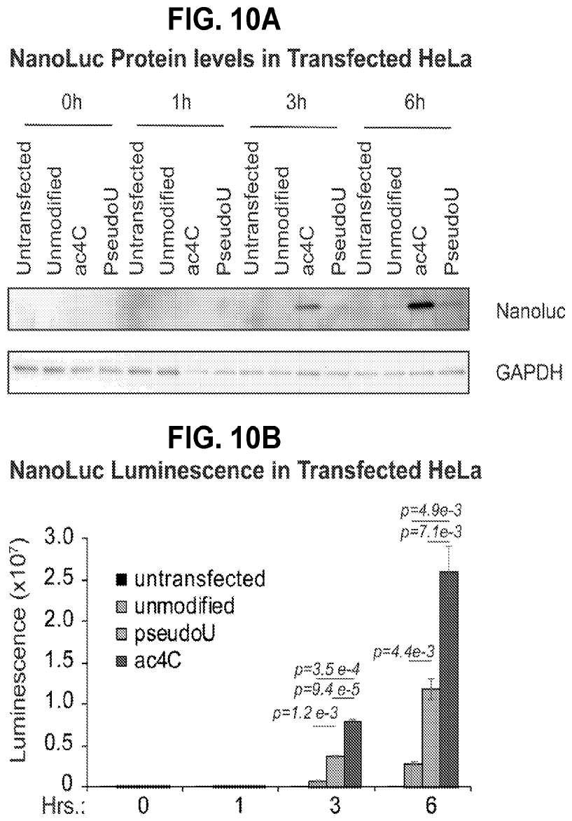

As shown in FIGS. 10A and 10B, ac4C stimulates mRNA translation as compared to unmodified mRNA and mRNA containing the uridine modification pseudouridine. Western blot for Nanoluciferase (Nanoluc) protein levels in HeLa cells transfected with in vitro transcribed Nanoluc mRNA with either canonical nucleotides, or NTPs in which ac4C or pseudouridine (PseudoU) have been substituted for C or U, respectively. Luminescence for Nanoluc in transfected HeLa cells with in vitro transcribed Nanoluc mRNA with either canonical nucleotides, or mixtures containing ac4C or PseudoU as in (10A).

### REFERENCES

1. Arango et al., Mol Cell 2022.
2. Arango et al., Cell 2018, 175, 1872-1886 e1824.
3. Kiss and Filipowicz, Genes Dev 1995, 9, 1411-1424.
4. Reichow et al., Nucleic Acids Res 2007, 35, 1452-1464.
5. Heiss et al., Methods Mol Biol 2021, 2298, 279-306.
6. Traube et al., Nat Protoc 2019, 14, 283-312.
7. Tyc and Steitz, EMBO J 1989, 8, 3113-3119.
8. Veitia, Exploring the etiology of haploinsufficiency. Bioessays 2022, 24, 175-184.
9. Fisher, and Scambler, Human haploinsufficiency—one for sorrow, two for joy. Nat Genet 1994, 7, 5-7.
10. Dang et al., Identification of human haploinsufficient genes and their genomic proximity to segmental duplications. Eur J Hum Genet 2008, 16, 1350-1357.
11. Collins et al., A cross-disorder dosage sensitivity map of the human genome. Cell 2022, 185, 3041-3055.e3025.
12. Claes et al., De Novo Mutations in the Sodium-Channel Gene SCN1A Cause Severe Myoclonic Epilepsy of Infancy. The American Journal of Human Genetics 2001, 68, 1327-1332.
13. Moumne et al., Oligonucleotide Therapeutics: From Discovery and Development to Patentability. Pharmaceutics 2022, 14.
14. Gagliardi, M., and Ashizawa, A. T. The Challenges and Strategies of Antisense Oligonucleotide Drug Delivery. Biomedicines 2021, 9.
15. Collins et al., Mild overexpression of MeCP2 causes a progressive neurological disorder in mice. Hum Mol Genet 2004, 13, 2679-2689.

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. An engineered nucleotide sequence, comprising, 5′ to 3′:
(a) a first targeting sequence;
(b) a C box motif containing sequence;
(c) a second targeting sequence; and
(d) a D box motif containing sequence.

2. The engineered nucleotide sequence of claim 1, wherein the first targeting sequence comprises about 15 to about 30 nucleotides.

3. The engineered nucleotide sequence of claim 1, wherein the second targeting sequence comprises about 15 to about 30 nucleotides.

4. The engineered nucleotide sequence of claim 1, wherein the first targeting sequence and the second targeting sequence are complementary to separate regions of a target sequence.

5. The engineered nucleotide sequence of claim 4, wherein the target sequence comprises an RNA transcript.

6. The engineered nucleotide sequence of claim 5, wherein the target sequence of the RNA transcript comprises a 5′ untranslated region of the RNA transcript.

7. The engineered nucleotide sequence of claim 5, wherein the target sequence of the RNA transcript comprises a coding sequence of the RNA transcript.

8. The engineered nucleotide sequence of claim 4, wherein the target sequence comprises a known acetylation site or a putative acetylation site, optionally wherein the target sequence is within about 30-50 nucleotides of the known acetylation site or the putative acetylation site.

9. The engineered nucleotide sequence of claim 4, wherein the C box motif containing sequence comprises a 5′ RUGAUGA-3′ sequence (wherein R is a purine).

10. The engineered nucleotide sequence of claim 1, wherein the C box motif containing sequence comprises about 40 to about 200 nucleotides.

11. The engineered nucleotide sequence of claim 1, wherein the D box motif containing sequence comprises a 5′ CUGA-3′ sequence.

12. The engineered nucleotide sequence of claim 1, wherein the D box motif containing sequence comprises about 15 to about 50 nucleotides.

13. The engineered nucleotide sequence of claim 1, wherein the engineered nucleotide sequence recruits NAT10 to the target sequence and causes acetylation (e.g., N4-acetylcytidine (ac4C) modification) of the target sequence.

14. The engineered nucleotide sequence of claim 1, wherein the targeting sequences are complementary to a target sequence in a desired target gene, and the gene is involved or implicated in any disease or disorder where moderate protein expression can lead to alleviation of the disease or disorder phenotype.

15. The engineered nucleotide sequence of claim 14, wherein the desired target gene encodes a ribosomal protein, a gene involved in RNA processing, a gene involved in gene transcription and/or translation, or a gene involved in proteostasis.

16. The engineered nucleotide sequence of claim 15, wherein the desired target gene is selected from the group consisting of CD25, CHD2, CFTR, CHD8, COL1A1, COL1A2, FBN1, FIGLA, FOXG1, FOXP2, FZD4, GATA2, GATA4, GI12, GLI3, HNF1B, HNF4A, HOXD13, IKZF1, IL2RA, IRF6, KIF1B, KIF21A, MECP2, MED13L, MEF2C, MEIS2, NHF1B, NKX2-5, NSD1, NR2F2, NR5A1, PAX2, PAX3, PAX5, PAX6, PBX1, PITX2, PPARG, POU3F2, RAI1, RUNX1, RUNX2, SCN1A, SETBP1, SETD1A, SIM, SIX3, SLC52A2, SOX2, SOX9, SOX10, TBX1, TBX5, TBR1, ZIC2, and ZFPM2.

17. The engineered nucleotide sequence of claim 1, wherein the targeting sequences are complementary to a target sequence in a desired target gene, and the desired target gene is involved or implicated in cancer.

18. The engineered nucleotide sequence of claim 17, wherein the desired target gene is selected from the group consisting of AKT1, ALK, APC, ATM, BAG1, BARD1, BMPR1A, BRAF, BRCA1, BRCA2, BRIP1, CDK4, CDH1, CDK4, CDKN1C, CDKN2A, CHEK2, CHK2, CREBBP, CTNNB1, EGFR, EP300, EPCAM, ERBB2, ETV6, FBXW7, FGFR2, FHIT, FLT3, FOXL2, GNAQ, GNAS, HOXB13, HRAS, KIT, KRAS, MAP2K1, MET, MLH1, MLL, MSH2, MSH6, MYC, NF1, NTR3, NTRK1, NRAS, PALB2, PAX8, PDGFRA, PIK3CA, PMS2, PPARγ, PRCC, PRKAR1A, PTEN, RAD51D, RET, RUNXBP2, SMAD4, SRC, STK11, TFE3, TGF-β, TGF-βRII, TP53, VEGFA, and WWOX.

19. A vector, comprising the engineered nucleotide sequence of claim 1.

20. (canceled)

21. A pharmaceutical composition, comprising the engineered nucleotide sequence of claim 1 and a pharmaceutically acceptable excipient.

22. A method for directing acetylation to a target sequence, wherein the method comprises administering the engineered nucleotide sequence of claim 1.

23. A method for treating a disease or disorder, wherein the method comprises administering the engineered nucleotide sequence of claim 1 to a subject in need thereof.

24-32. (canceled)


SEQUENCE LISTING (10 sequences)

SEQ ID NO: 1 - DNA, 105 bp, Synthetic construct
  source 1..105: mol_type = other DNA
gtccgaccttcaccttccgagcgtgatgattgggtgttcatacgcttgtgtgagatgtgccaccctttgtctgagcgatgtggtgggcacattacccgtctgacc

SEQ ID NO: 2 - DNA, 20 bp, Synthetic construct
  source 1..20: mol_type = other DNA
agacatccaaggaaggttta

SEQ ID NO: 3 - DNA, 16 bp, Synthetic construct
  source 1..16: mol_type = other DNA
acatccaaggaaggtt

SEQ ID NO: 4 - DNA, 20 bp, Synthetic construct
  source 1..20: mol_type = other DNA
taaggaaaacctttctggtg

SEQ ID NO: 5 - DNA, 20 bp, synthetic construct
  source 1..20: mol_type = other DNA
ttctggtggaaactgcgaat

SEQ ID NO: 6 - DNA, 20 bp, synthetic construct
  source 1..20: mol_type = other DNA
ctgcgaatgttggaaaacca

SEQ ID NO: 7 - DNA, 20 bp, synthetic construct
  source 1..20: mol_type = other DNA
tcactgtgatgatggttttc

SEQ ID NO: 8 - DNA, 20 bp, synthetic construct
  source 1..20: mol_type = other DNA
ctcactcagacatccaagga

SEQ ID NO: 9 - RNA, 105 nt, synthetic construct
  source 1..105: mol_type = other RNA
gtccgaccttcaccttccgagcgtgatgattgggtgttcatacgcttgtgtgagatgtgccaccctttgtctgagcgatgtggtgggcacattacccgtctgacc

SEQ ID NO: 10 - RNA, 38 nt, synthetic construct
  source 1..38: mol_type = other RNA
cacatcgctcagacaccatggggaaggtgaaggtcgga